(19) United States
(12) Patent Application Publication
(10) Pub. No.: US 2006/0224138 A1
(43) Pub. Date: Oct. 5, 2006

(54) Disposable garment

(75) Inventors: Yukio Heki, Kobe (JP); Hideaki Sasaki, Nishinomiya (JP); Hiroyuki Ueno, Kobe (JP)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company

(21) Appl. No.: 11/397,052

(22) Filed: Apr. 3, 2006

Related U.S. Application Data
(60) Provisional application No. 60/668,198, filed on Apr. 4, 2005.

(51) Int. Cl. A61F 13/15

(52) U.S. Cl. 604/385.31; 604/385.28

(57) ABSTRACT

A disposable garment used with an insert pad. The disposable garment has a longitudinal centerline, a transverse centerline, a crotch region, a front region and a back region. The disposable garment comprises an outer cover and an absorbent assembly joined inside the outer cover. The absorbent assembly has a longitudinal centerline, a transverse centerline, longitudinal sides and transverse ends. The absorbent assembly comprises a backsheet, an absorbent material disposed inside the backsheet and an elastic member extending in the longitudinal direction of the absorbent assembly to longitudinally elasticize a portion of the absorbent assembly. The absorbent assembly has a center crotch portion and a periphery portion, each portion having an absorbent capacity per unit area. The absorbent capacity per unit area of the center crotch portion is less than that of the periphery portion. The absorbent assembly is treated to increase stiffness.

### CROSS REFERENCE TO RELATED APPLICATION

This application claims the benefit of U.S. Provisional Application No. 60/668,198, filed Apr. 4, 2005.

### FIELD OF THE INVENTION

The present invention relates to a disposable garment which may be used with an insert pad.

### BACKGROUND OF THE INVENTION

Infants and other incontinent individuals wear disposable absorbent articles such as diapers to receive or contain urine and other body exudates. The diapers are typically worn directly on the wearer's skin to directly receive or contain body exudates. Insert pads used with diapers have been also known. Since the insert pad is placed inside the diaper and absorbs or contains body exudates, the diaper could remain clean even after body exudates are discharged and be reusable by exchanging only the insert pad. While such a system is economical since the diaper may be repeatedly used, the wearer feels uncomfortableness due to bulkiness of the combination of the diaper and the insert pad. It also causes problems such as deterioration of fitment, deterioration of support of the insert pad or leakage of body exudates due to the fitment and/or support deterioration.

Conventional diapers typically had an absorbent material at least at the center of the diaper since the center of the diaper is the portion in which body exudates are most frequently discharged. However, the absorbent material at the center of the diaper is actually unnecessary when the diaper is used with an insert pad which functions to absorb or contain body exudates. Therefore, attempts have been made to remove the absorbent material at the center of the diaper to reduce the bulkiness of the combination of the diaper and the insert pad. It not only resolves the problems associated with the bulkiness of the diaper/insert pad combination but also reduces the cost of manufacturing the diaper because of less absorbent material required. However, removal of the absorbent material causes other drawbacks to be resolved. Namely, the diaper from which the absorbent material is removed is less stiff than the diaper from which the absorbent material is not removed and makes it difficult or complicated to handle the semi-assembled diaper during conveyance of the semi-assembled diaper in the manufacturing process. Such drawbacks are particularly remarkable in a disposable garment which is formed with an outer cover and an absorbent assembly comprising an absorbent material and an elastic member such as an elasticized barrier leg cuff or an elasticized outer leg cuff. In this type of disposable garment, the outer cover and the absorbent assembly are typically separately formed and then they are combined to one another. When forming the absorbent assembly separately, the contract force of the elastic member of the absorbent assembly tends to contract or bend the absorbent assembly by overcoming the less stiff absorbent assembly. Therefore, it makes it difficult or complicated to handle the less stiff absorbent assembly before, when or after joining the absorbent assembly to the outer cover in the manufacturing process. Alternatively, after the absorbent assembly is joined to the outer cover by, e.g., adhesives, the less stiff absorbent assembly could be detached from the outer cover because the less stiff absorbent assembly could be contracted or bent by the elastic member.

Based on the foregoing, there is a need for an economical disposable garment which is used with an insert pad.

There is also a need for a disposable garment which is able to improve fitment, to support an insert pad or to reduce leakage of body exudates.

There is also a need for a disposable garment which allows easy handling of the disposable garment in the manufacturing process.

### SUMMARY OF THE INVENTION

The present invention is directed to a disposable garment. The disposable garment has a longitudinal centerline, a transverse centerline, a crotch region, a front region and a back region. The disposable garment comprises an outer cover and an absorbent assembly joined inside the outer cover. The absorbent assembly has a longitudinal centerline, a transverse centerline, longitudinal sides and transverse ends. The absorbent assembly comprises a backsheet, an absorbent material disposed inside the backsheet and an elastic member extending in the longitudinal direction of the absorbent assembly to longitudinally elasticize a portion of the absorbent assembly. The absorbent assembly has a center crotch portion and a periphery portion, each portion having an absorbent capacity per unit area. The absorbent capacity per unit area of the center crotch portion is less than that of the periphery portion. The absorbent assembly is treated to increase stiffness.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings in which like designations are used to designate substantially identical elements, and in which:

FIG. 1 is a perspective view of the disposable garment of the present invention in a typical in-use configuration;

FIG. 2 is a top plan view of the disposable garment in its flat uncontracted condition showing the inner surface with a portion of elements cut out;

FIG. 3 is a cross-sectional view of FIG. 2 taken along the line III-III;

FIG. 4 is a cross-sectional view of FIG. 2 taken along the line IV-IV;

FIG. 5 is a schematic top plan view of the disposable garment in its flat uncontracted condition showing the inner surface with various elements being abbreviated;

FIG. 6 is a cross-sectional view of the disposable garment with an insert pad placed thereon in a typically in-use configuration taken along the line corresponding to the line III-III in FIG. 2;

FIGS. 7-10 are a schematic top plan view showing various dispositions of the absorbent material;

FIGS. 11-14 are a schematic top plan view showing various dispositions of the area to be treated to increase stiffness;

FIGS. 15 and 16 are a schematic top plan view of an alternative embodiment showing disposition of the outer cover elastic member in relation to the absorbent material;

FIG. 17 is a schematic top plan view of the disposable garment showing the directions for using an insert pad;

FIG. 18 is a schematic view showing the process for forming the disposable garment shown in FIG. 1; and

FIG. 19 is a schematic top plan view of an alternative embodiment of the outer cover.

### DETAILED DESCRIPTION OF THE INVENTION

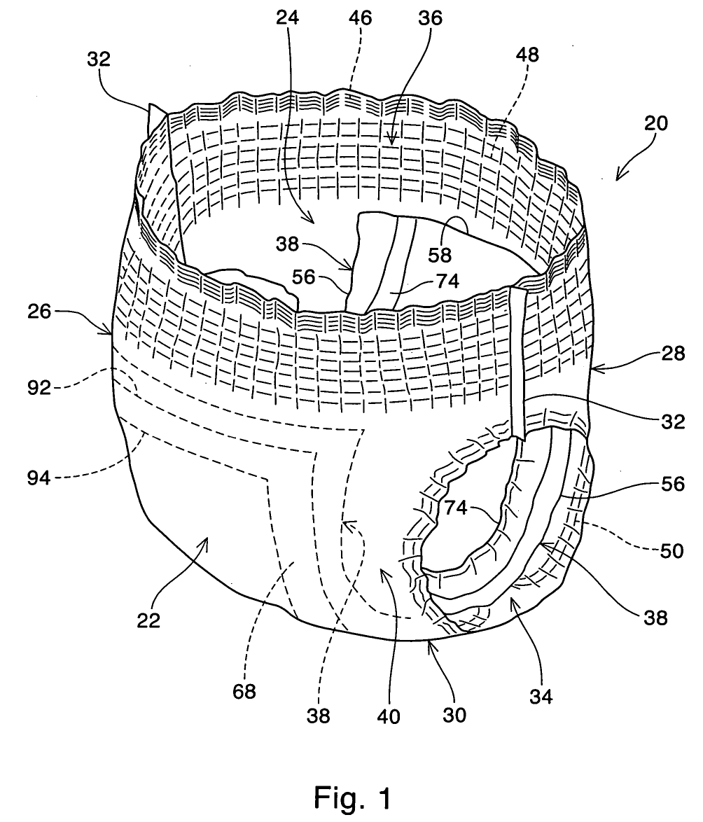

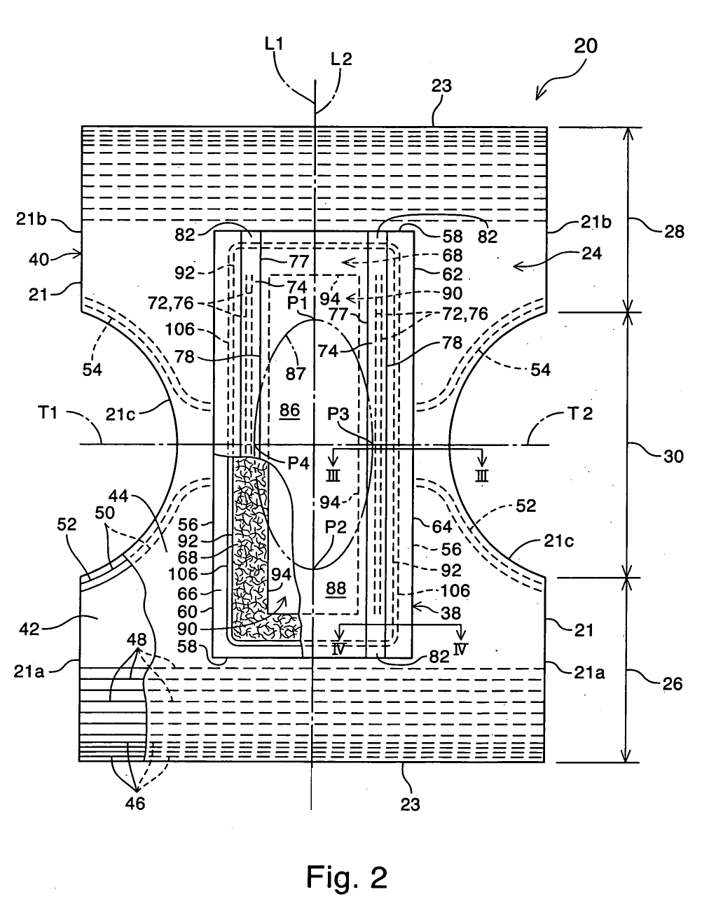

FIG. 1 is a perspective view of the disposable garment 20 of the present invention. As used herein, the term “disposable” is used herein to describe garments which are not intended to be laundered but which could be reused before getting soiled (i.e., they are intended to be discarded after getting soiled). The disposable garment 20 shown in FIG. 1 is formed into a pull-on garment. The term “pull-on garment” refers to articles of wear which have a defined waist opening and a pair of leg openings and which are pulled onto the body of the wearer by inserting the legs into the leg openings and pulling the article up over the waist. Alternatively, the disposable garment 20 may be formed into an open type garment in which the opposite longitudinal side edges are not joined before use of the garment but may be joined by a fastening means after the garment is worn on the wearer. The pull-on garment 20 has an outer surface 22, an inner surface 24 opposed to the outer surface 22, a front region 26, a back region 28, a crotch region 30, and seams 32 which join the front region 26 and the back region 28 to form leg openings 34 and a waist opening 36. The garment 20 also has a longitudinal centerline L1, a transverse centerline T1, longitudinal side edges 21, transverse end edges 23 as shown in FIG. 2. The longitudinal side edge 21 comprises a front straight longitudinal side edge 21a, a back straight longitudinal side edge 21b and a concave curved side edge 21c. The garment 20 comprises an outer cover 40 and an absorbent assembly 38 joined inside the outer cover 40.

The outer cover 40 covers the wearer's body, specifically the front and back waist region and the crotch region of the wearer. The outer cover 40 acts to dynamically create fitment forces and to distribute the forces dynamically generated during wear such that the garment 20 snugly fits the wearer. The outer cover 40 comprises two layers and elastic members (outer cover elastic members) interposed between the two layers. The two layers comprise the outer layer 42 disposed on the outer surface 22 of the garment 20 and the inner layer 44 disposed on the inner surface 24 of the garment 20. The outer cover elastic members comprise the waist end elastic member 46, the waist body elastic member 48 and the leg elastic member 50.

The outer layer 42 and the inner layer 44 may have any shape being capable of covering the wearer's body. In the embodiment shown in FIG. 2, the outer layer 42 and the inner layer 44 are coextensive and have the same rectangular shape. Alternatively, they may have an hourglass shape. The outer and inner layers 42, 44 may comprise any known materials. Suitable material for the outer and inner layers 42, 44 can be manufactured from a wide range of materials such as plastic films; apertured plastic films; woven or nonwoven webs of natural materials (e.g., cotton fibers), synthetic fibers (e.g., polyolefins, polyamides, polyester, polyethylene, or polypropylene fibers), or a combination of natural and/or synthetic fibers; or coated woven or nonwoven webs. Preferably the layer 42, 44 comprises a nonwoven web of synthetic fibers. The layer 42, 44 may comprise a stretchable material such as a stretchable nonwoven. If the layer 42, 44 comprises a stretchable material, the outer cover elastic members may not be necessary since the stretchable material of the layer 42, 44 could provide fitment forces and either of the outer layer 42 or the inner layer 44 may not be necessary since it is not necessary to sandwich the outer cover elastic members between the outer layer 42 and the inner layer 44. Alternatively, the outer and inner layers 42, 44 may comprise a hydrophobic, non-stretchable nonwoven material. Alternatively, the outer layer 42 and the inner layer 44 may have different shapes and/or may be formed with different materials. The outer and inner layer 42, 44 may comprise a single piece of material or may comprise a plurality of pieces of material which are joined each other.

The outer cover elastic members comprise the waist end elastic member 46, the waist body elastic member 48 and the leg elastic member 50. The waist end elastic member 46, the waist body elastic member 48 and the leg elastic member 50 may comprise one or more of elastic elements such as strands or panels. The waist end elastic member 46 and the waist body elastic member 48 are disposed to extend in the transverse direction along the transverse end edge 23. The waist end elastic member 46 is disposed along the transverse end edge 23. The leg elastic member 50 is disposed about the leg opening 34. In the embodiment shown in FIG. 2, the waist end elastic member 46 and the waist body elastic member 48 comprise a plurality of elastic strands which are disposed at a constant interval in the longitudinal direction. Alternatively, the waist end elastic member 46 and the waist body elastic member 48 may be disposed at a different interval in the longitudinal direction. The leg elastic member 50 comprises a front leg elastic 52 and the back leg elastic 54. The front leg elastic 52 extends from the intersection of the front straight longitudinal side edge 21a and the concave curved side edge 21c toward the transverse centerline T1 along the curved side edge 21c, curves toward the transverse direction before reaching the transverse centerline T1 and then terminates before overlapping the absorbent assembly 38. The back leg elastic 54 also has the same structure. Therefore, the front and back leg elastics 52, 54 do not overlap in the crotch region 30. The outer cover elastic members are interposed between the outer layer 42 and the inner layer 44 and joined therebetween in a stretched condition of the outer cover elastic member such that the outer cover 40 provides elasticity when the garment 20 is worn.

The absorbent assembly 38 is joined inside and entirely covered by the outer cover 40 such that the absorbent assembly 38 covers the crotch region of the wearer. In the embodiment shown in FIG. 2, the absorbent assembly 38 has a generally rectangular shape having a longitudinal centerline L2, a transverse centerline T2, longitudinal sides 56 and transverse ends 58. The absorbent assembly 38 also has a front waist panel 60 positioned in the front region 26 of the garment 20, a back waist panel 62 positioned in the back waist region 28, and a crotch panel 64 in the crotch region 30. The absorbent assembly 38 does not overlap with the waist body elastic member 48 and the leg elastic member 50. Alternatively, the absorbent assembly 38 may overlap therewith.

The absorbent assembly 38 comprises a backsheet 66 which may be liquid impervious and an absorbent material 68 disposed inside the backsheet 66. The absorbent assembly 38 also comprises an elastic member 72 (absorbent assembly elastic member) extending in the longitudinal direction of the absorbent assembly 38 to longitudinally elasticize a portion of the absorbent assembly 38. The absorbent assembly 38 may comprise a topsheet 70 which may be liquid pervious to sandwich the absorbent material 68 between the topsheet 70 and the backsheet 66.

The backsheet 66 is generally that portion of the absorbent assembly 38 positioned adjacent the garment-facing surface of the absorbent assembly 38. The backsheet 66 prevents the exudates absorbed and contained therein from soiling articles that may contact the garment 20, such as bedsheets and undergarments. In preferred embodiments, the backsheet 66 is impervious to liquids (e.g., urine) and comprises a thin plastic film. Suitable backsheet materials may include breathable materials that permit vapors to escape from the garment 20 while still preventing exudates from passing through the backsheet 66.

The topsheet 70 is preferably positioned adjacent the body-facing surface of the absorbent material 68 and may be joined thereto and/or to the backsheet 66 by any attachment means known in the art. The topsheet 70 is preferably compliant, soft feeling, and non-irritating to the wearer's skin. Further, at least a portion of the topsheet 70 is liquid pervious, permitting liquid to readily penetrate through its thickness. A suitable topsheet 70 may be manufactured from a wide range of materials, such as porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. Preferably, the topsheet 70 is made of a hydrophobic material or is treated to be hydrophobic in order to isolate the wearer's skin from liquids contained in the absorbent material 68. If the topsheet 70 is made of a hydrophobic material, preferably at least the upper surface of the topsheet 70 is treated to be hydrophilic so that liquids will transfer through the topsheet more rapidly.

The absorbent assembly elastic member 72 extends in the longitudinal direction of the absorbent assembly 38. The absorbent assembly elastic member 72 may be disposed along or adjacent the longitudinal side 56 of the absorbent assembly 38. The absorbent assembly elastic member 72 longitudinally elasticizes a portion of the absorbent assembly 38. The absorbent assembly elastic member 72 may comprise any longitudinally extending elastic member. For example, the absorbent assembly elastic member 72 may be an elastic member of a barrier leg cuff if the absorbent assembly 38 is provided with a barrier leg cuff. Alternatively, the absorbent assembly elastic member 72 may be an elastic member of an elasticized outer leg cuff if the absorbent assembly 38 is provided with an elasticized outer leg cuff along the longitudinal side 56. In the embodiment shown in FIGS. 2, 3 and 4, the absorbent assembly 38 is provided with a barrier leg cuff 74 adjacent the longitudinal side 56 of the absorbent assembly 38. The barrier leg cuff 74 provides improved containment of liquids and other body exudates in the crotch region 30. The barrier leg cuff 74 comprises a single layer of material which is folded into two layers. The barrier leg cuff 74 extends from the longitudinal side 56 toward the longitudinal centerline L2 and then is folded along the folding line 77 back toward the longitudinal side 56. The barrier leg cuff 74 has a distal edge 78 and a proximal edge 80. The barrier leg cuff 74 has a barrier cuff elastic member 76 disposed at the distal edge 78. The proximal edge 80 of the barrier leg cuff 74 is joined to the backsheet 66 adjacent the longitudinal side 56. The portion of the barrier leg cuff 74 along the folding line 77 and the distal edge 78 are free from attachment to any portion of the absorbent assembly 38 in the crotch region 30 such that the barrier leg cuff 74 stands up toward the wearer's body when the garment 20 is used. In this embodiment, the barrier leg cuff elastic member 76 (i.e., the absorbent assembly elastic member 72) longitudinally elasticizes a portion of the absorbent assembly 38 (i.e., the barrier leg cuff 74 constituting a portion of the absorbent assembly 38). The transverse end 82 of the barrier leg cuff 74 is joined to the topsheet 70 adjacent the folding line 77 by an attachment means 84 such as an adhesive and is joined to the barrier leg cuff 74 itself adjacent the distal edge 78 by an attachment means 84 such as an adhesive as shown in FIG. 4. While the barrier leg cuff 74 is folded along the folding line 77 back toward the longitudinal side 56, the barrier leg cuff 74 may not be folded and may extend toward the longitudinal centerline L1.

The absorbent assembly 38 has a center crotch portion 86 and a periphery portion 88. In FIG. 2, the center crotch portion 86 is encircled by an imaginary line of an alternate long and two short dashes line 87. The center crotch portion 86 is the portion where body exudates are typically loaded if the disposable garment 20 is directly worn on the wearer. The center crotch portion 86 is the portion which an insert pad is placed on and at least intends to cover when the insert pad is used. The center crotch portion 86 can be determined by the shape and the area occupied in the absorbent assembly 38. The center crotch portion 86 may have an oval shape having a transverse axis and a longitudinal axis. The width B of the transverse axis is defined by the distance between the point P3 and the point P4 on the transverse centerline T2. The length A of the longitudinal axis is defined by the distance between the point P1 and the point P2 on the longitudinal centerline L2. The ratio of A:B of the center crotch portion 86 may be between 1:1 and 3.2:1. The area of the center crotch portion 86 may be between 20% and 50% of the area of the absorbent assembly 38 when the absorbent assembly is stretched in its flat uncontracted condition. For the determination of the center crotch portion 86, the ratio of A:B being 1.6:1 and the area being 35% of the area of the absorbent assembly may be representatively used. The rest of the portion of the absorbent assembly 38 other than the center crotch portion 86 is the periphery portion 88.

Each of the center crotch portion 86 and the periphery portion 88 has an absorbent capacity per unit area. The absorbent material 68 is provided such that the absorbent capacity per unit area of the center crotch portion 86 is less than that of the periphery portion 88. The absorbent capacity per unit area of the center crotch portion 86 may be not more than 40%, preferably not more than 30%, more preferably not more than 20%, of the absorbent capacity per unit area of the periphery portion 88. The center crotch portion 86 may have no absorbent material to have no absorbent capacity. The absorbent capacity per unit area is measured by the method described below. Since the center crotch portion 86 is intended to be covered by an insert pad and the insert pad is expected to receive and contain body exudates, the center crotch portion 86 does not need to have much absorbent capacity per unit area. The center crotch portion 86 having low absorbent capacity per unit area contributes to reduce bulkiness in the crotch region when the insert pad is used with the disposable garment 20 because low absorbent capacity per unit area can be typically achieved by reducing the amount of the absorbent material 68. It also contributes to reduce the cost of the material used for the absorbent material 68. The absorbent capacity per unit area can be adjusted by, e.g., changing the basis weight of the absorbent material 68 and/or the area in which the absorbent material 68 is present such that the absorbent capacity per unit area of the center crotch portion 86 is less than that of the periphery portion 88. In one embodiment, the basis weight of the absorbent material 68 may be less in the center crotch portion 86 than in the periphery portion 88 if the both portions 86 and 88 use the same absorbent material. For example, the basis weight of the center crotch portion 86 may be not more than 50%, preferably not more than 40%, more preferably not more than 30%, of the basis weight of the periphery portion 88. In another embodiment, the ratio of the absorbent material area (which means the area in which the absorbent material 68 is present in the center crotch portion 86) occupied in the area of the center crotch portion 86 may be less than the ratio of the absorbent material area (which means the area in which the absorbent material 68 is present in the periphery portion 88) occupied in the area of the periphery portion 88. The ratio can be obtained by:

(absorbent material area)/(area of center crotch portion), and

(absorbent material area)/(area of periphery portion).

For example, the ratio of the absorbent material area occupied in the area of the center crotch portion 86 may be not more than 0.4, preferably not more than 0.3, more preferably not more than 0.2. The center crotch portion 86 may not have any absorbent material, in which case the ratio is zero. On the contrary, the periphery portion 88 has more amount of the absorbent material 68 than the center crotch portion 86. The ratio of the absorbent material area occupied in the area of the periphery portion 88 may be not less than 0.6, preferably not less than 0.65, more preferably not less than 0.7.

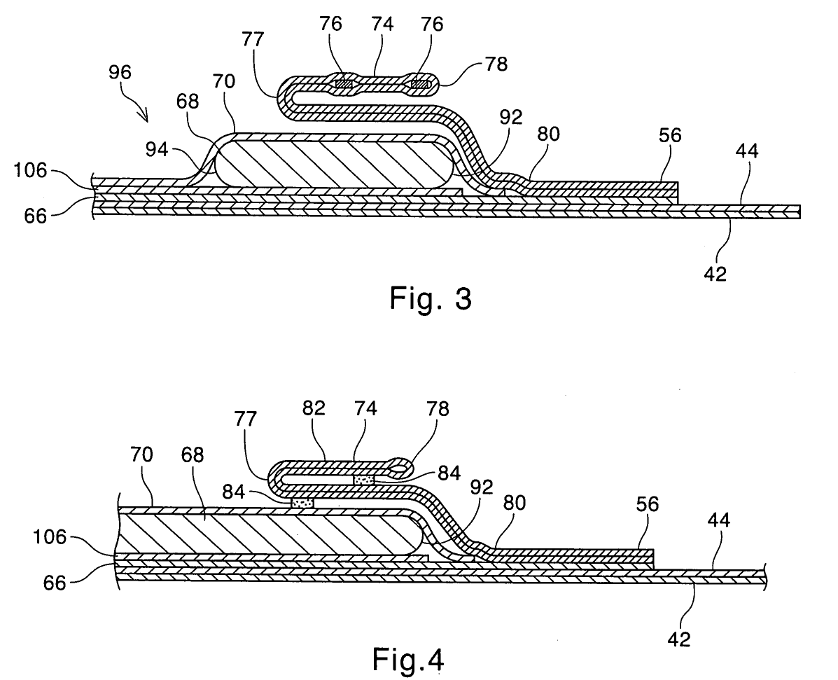

The gradient of the absorbent material amount is provided between the center crotch portion 86 and the periphery portion 88 and the concave portion 96 is formed in the absorbent assembly 38 (refer to FIG. 3). The presence of the absorbent material 68 disposed in the periphery portion 88 is beneficial to provide the user with a sense of security of preventing leakage of body exudates. It is preferable to dispose the absorbent material 68 to continuously surround the center crotch portion 86. Alternatively, the absorbent material 68 may be discontinuously disposed in the periphery portion 88.

The absorbent assembly 38 has a concave portion 96 in cross-sectional view to receive a portion of or a majority portion of the insert pad when the insert pad is placed on the garment 20. The size of the insert pad may be designed to be typically bigger than the area of the center crotch portion 86 so that the insert pad receives and contains body exudates and does not soil the disposable garment for repeated use. In order to accommodate the entirety of or the majority portion of the insert pad, the area of the concave portion 96 may be preferably bigger than the area of the center crotch portion 86. Alternatively, the area of the concave portion 96 may be smaller than the area of the center crotch portion 86 if desired.

In the embodiment shown in FIG. 2, the absorbent material 68 is disposed continuously and has a generally rectangular shape with an opening 90 having a generally rectangular shape. The absorbent material 68 has an outer edge 92 and an inner edge 94. The outer edge 92 defines the outer shape of the absorbent material 68 and the inner edge 94 defines the shape of the opening 90. The majority of the absorbent material 68 is disposed in the periphery portion 88 while a small amount of the absorbent material 68 is disposed in the center crotch portion 86 (in the embodiment shown in FIG. 2, a small amount of the absorbent material 68 is present in the center crotch portion 86 along a portion of the opposite longitudinal sides of the longitudinally extending oval shape defined by the line 87). Alternatively, the absorbent material 68 may be disposed only in the periphery portion 88 and may not be disposed in the center crotch portion 86.

The absorbent assembly 38 is treated to increase stiffness thereof. The stiffened absorbent assembly 38 allows easy handling of the absorbent assembly 38 before, when or after joining the absorbent assembly 38 to the outer cover 40 in the manufacturing process since the stiffened absorbent assembly 38 tends to withstand the contract force of the absorbent assembly elastic member 72. The stiffened absorbent assembly 38 also helps the absorbent assembly 38 be kept attached to the outer cover 40.

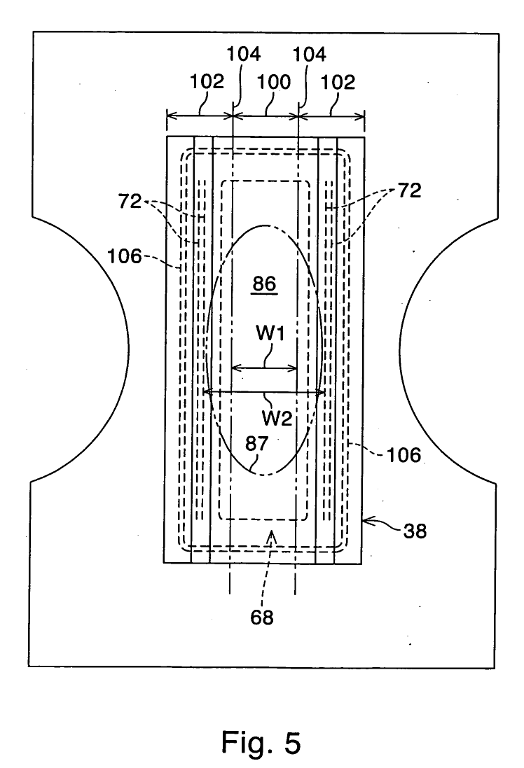

Referring to FIG. 5, the absorbent assembly 38 has a longitudinally extending center portion 100 and longitudinally extending opposite side portions 102 which are sectioned by an imaginary line of an alternate long and two short dashes line 104. The transverse width W1 of the longitudinally extending center portion 100 is not more than 40%, preferably not more than 30%, more preferably not more than 20%, of the transverse width W2 between the positions of the absorbent assembly elastic members 72 when the absorbent assembly 38 is stretched in its flat uncontracted condition. When one side of the absorbent assembly elastic member 72 comprises two or more elastic materials (in the embodiment shown in FIG. 5, one side of the absorbent assembly elastic member 72 comprises two elastic strands), the width W2 should be determined by the width between the transversely innermost elastic members. When the absorbent assembly 38 is stretched in its flat uncontracted condition against the absorbent assembly elastic member 72, the absorbent assembly elastic member 72 is positioned in the area of the longitudinally extending side portion 102 as shown in FIG. 5. Therefore, it is preferable to treat at least the longitudinally extending side portion 102 to increase stiffness thereof such that the stiffened portion of the longitudinally extending side portion 102 withstands the contraction force of the absorbent assembly elastic member 72. A portion of or the entirety of the longitudinally extending side portion 102 along its length may be treated to increase stiffness. A portion of or the entirety of the longitudinally extending center portion 100 may be additionally treated to increase stiffness.

The absorbent assembly 38 may be treated to increase stiffness with a variety of means. For example, the absorbent assembly 38 may be incorporated with a reinforcement member 106 to increase stiffness of the absorbent assembly member 38. In the embodiment shown in FIGS. 3 and 4, the reinforcement member 106 is disposed between the absorbent material 68 and the backsheet 66 and between the topsheet 70 and the backsheet 66 in the area where the opening 90 is present. Alternatively, the reinforcement member 106 may be disposed on the wearer facing side of the topsheet 70, on the garment facing side of the backsheet 66, or between any element constituting the absorbent assembly 38. The reinforcement member 106 extends in the almost entirety of the absorbent assembly 38 as shown in FIG. 2. In the embodiment shown in FIG. 2, the reinforcement member 106 is bigger than the outer edge 92 of the absorbent material 68. The reinforcement member 106 may comprise any material which is added to the absorbent assembly 38 and which increases stiffness of the absorbent assembly 38. Examples of the reinforcement member 106 may include a sheet like material comprising, tissues; porous foams; reticulated foams; apertured plastic films; non-apertured plastic film; or woven or nonwoven webs of natural fibers (e.g., wood or cotton fibers), synthetic fibers (e.g., polyester or polypropylene fibers), or a combination of natural and synthetic fibers. The reinforcement member 106 may include materials which may be used for the absorbent material, the topsheet material and/or the backsheet material. The reinforcement member 106 preferably comprises a hydrophobic material since the hydrophobic material tends to maintain stiffness even though it gets wet by body exudates. In the embodiment shown in FIG. 2, the reinforcement member 106 comprises a nonwoven material.

An element constituting the absorbent assembly 38 may be treated to increase stiffness of the element itself. Such an element to be stiffened includes, but not limited to, the absorbent material 68, the topsheet 70 and/or the backsheet 66. For example, the absorbent material 68 may be stiffened by compressing or embossing a portion of or the entirety of the absorbent material. The absorbent material 68 compressed or embossed may be further reinforced by adhesive means to maintain its compressed or embossed configuration even when the absorbent material 68 gets wet. Alternatively, compression or embossment of the absorbent material 68 may be released when the absorbent material 68 gets wet such that the absorbent loses stiffness. This allows the absorbent assembly 38 to withstand the contraction force of the absorbent assembly elastic member 72 yet to provide softness to the wearer when the absorbent material 68 gets wet.

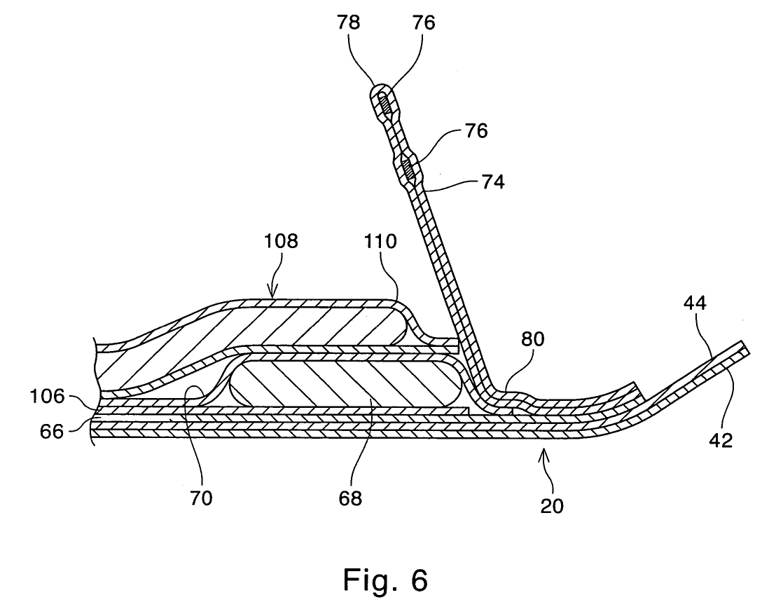

As shown in FIG. 6, an insert pad 108 is placed inside the disposable garment 20 such that the majority portion of the insert pad 108 is placed in the center crotch portion 86. The insert pad 108 may extend between the walls of the barrier leg cuffs 74 transversely and between the transverse end 58 of the absorbent assembly 38. The periphery 110 of the insert pad 108 may be superposed on the absorbent material 68. Alternatively, the insert pad 108 may be placed to fit into the concave portion 96. The insert pad 108 may comprise a topsheet, a backsheet and an absorbent material interposed therebetween. The insert pad 108 may also comprise a barrier leg cuff or an elasticized outer leg cuff.

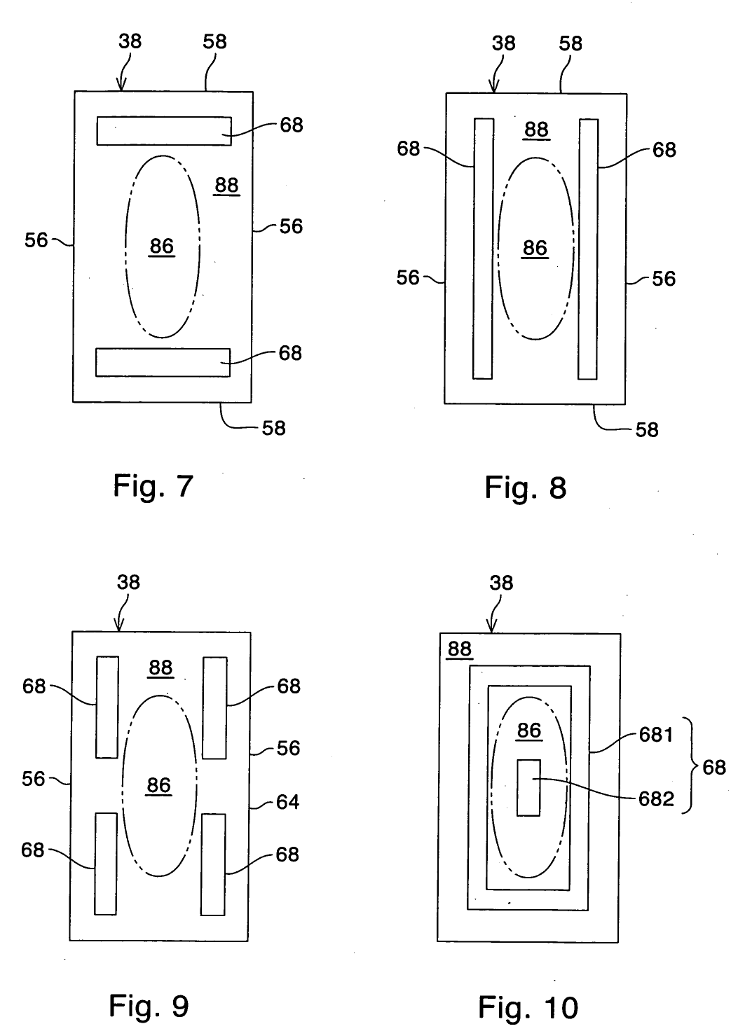

A variety of variations are possible with respect to the structure of the disposable garment 20. FIGS. 7-10 show various dispositions of the absorbent material 68. While the embodiment shown in FIGS. 1 and 2 has the absorbent material 68 continuously disposed to surround the center crotch portion 86, the embodiment shown in FIGS. 7-9 has the absorbent material 68 discontinuously disposed in the periphery portion 88 to partly surround the center crotch portion 86. The center crotch portion 86 has no absorbent material to have no absorbent capacity in FIGS. 7-9. The absorbent material 68 in FIG. 7 is disposed only along the transverse ends 58, but not disposed along the longitudinal sides 56. In contrast, the absorbent material 68 in FIG. 8 is disposed only along the longitudinal sides 56, but not disposed along the transverse ends 58. While FIG. 9 is the same as FIG. 8 in that the absorbent material 68 is disposed only along the longitudinal sides 56, they are different in that the absorbent material 68 is not disposed at the crotch panel 64 in the periphery portion 88 in FIG. 9. The absorbent material 68 in FIG. 10 comprises a first absorbent material 681 and a second absorbent material 682. The disposition of the first absorbent material 681 is generally the same as that in FIG. 2 such that the first absorbent material 681 is continuously disposed in the periphery portion 88. In the embodiment shown in FIG. 10, however, the second absorbent material 682 is provided around the center of the center crotch portion 86. The ratio of the absorbent material area occupied in the area of the center crotch portion 86 is less than the ratio of the absorbent material area occupied in the area of the periphery portion 88. The second absorbent material 682 helps the insert pad placed inside the disposable garment 20 keep contacting the source of the wearer's body discharging body exudates because the second absorbent material 682 biases the insert pad toward the wearer's body. In any embodiment, the absorbent material 68 is disposed such that the absorbent capacity per unit area of the center crotch portion 86 is less than that of the periphery portion 88.

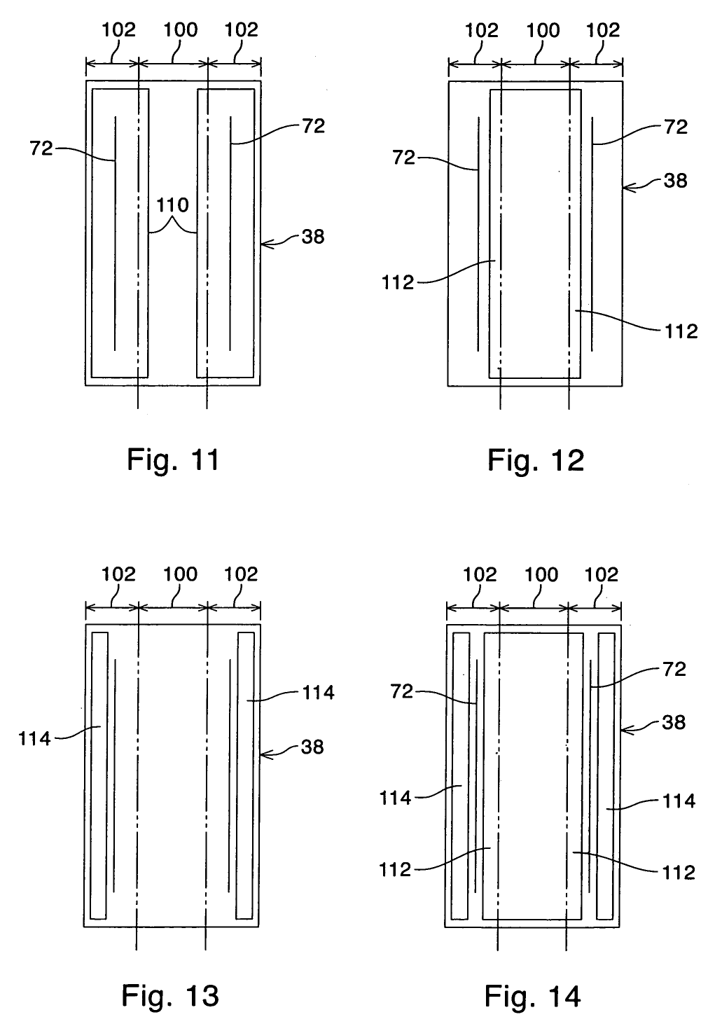

FIGS. 11-14 show various dispositions of the area to be treated to increase stiffness. In FIG. 11, the entirety of the longitudinally extending side portions 102 of the absorbent assembly 38 and the opposite sides 110 of the longitudinally extending center portion 100 are treated to increase stiffness. Since the entirety of the longitudinally extending side portion 102 is treated, the portion where the absorbent assembly elastic member 72 is present is stiffened. In FIG. 12, the inner sides 112 of the longitudinally extending side portion 102 and the entirety of the longitudinally extending center portion 100 are treated to increase stiffness. Alternatively, the entirety of the longitudinally extending center portion 100 may not be treated to increase stiffness. In FIG. 13, the outer sides 114 of the longitudinally extending side portions 102 are treated to increase stiffness. In FIGS. 12 and 13, the portion of the absorbent assembly elastic member 72 may be also treated to increase stiffness. In FIG. 14, the inner sides 112 and the outer sides 114 of the longitudinally extending side portions 102 and the entirety of the longitudinally extending center portion 100 are treated to increase stiffness. While the portion of the absorbent assembly elastic member 72 is not treated to increase stiffness in FIGS. 12, 13 and 14, the treatment to the inner sides 112 and/or the outer sides 114 of the longitudinally extending side portion 112 helps increase stiffness of the absorbent assembly 38. The treatment to increase stiffness of the absorbent assembly 38 may be made by incorporating the reinforcement member 106 or by treating the elements constituting the absorbent assembly 38 in the portion of the absorbent assembly 38 to be treated as described above.

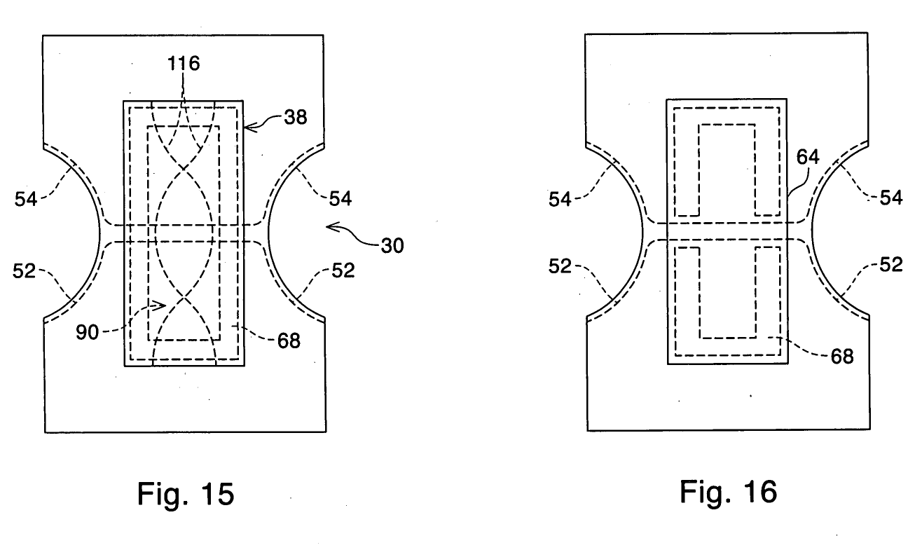

FIGS. 15 and 16 show an alternative embodiment of the outer cover elastic member. In FIG. 15, the front and back leg elastic members 52, 54 extend continuously across the crotch region 30. The continuous leg elastic members 52, 54 help support the insert pad toward the wearer's body when the insert pad is placed inside the disposable garment 20. In the embodiment shown in FIG. 15, the absorbent material 68 is continuously disposed to form the opening 90. The insert pad placed in the opening 90 is directly supported by the continuous leg elastic members 52 and 54. The outer cover elastic member in FIG. 15 may further comprise generally longitudinally extending elastic member 116 disposed in the area of the opening 90. The longitudinally extending elastic member 116 also helps directly support the insert pad toward the wearer's body. In FIG. 16, the front and back leg elastic members 52, 54 also extend continuously across the crotch region 30. However, the absorbent material 68 is discontinuous and is not present in the crotch panel 64 such that the absorbent material does not interfere the force of the front and back leg elastic members 52, 54.

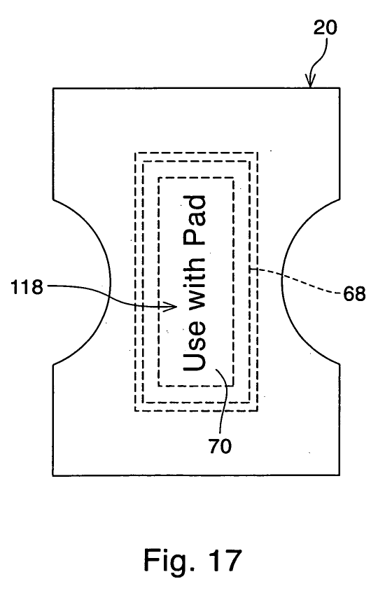

FIG. 17 shows an alternative embodiment of the disposable garment 20. The disposable garment 20 has an absorbent material 68 continuously disposed in the periphery portion (not shown in FIG. 17). Since the absorbent material is disposed such that the absorbent capacity per unit area of the center crotch portion 86 is less than that of the periphery portion 88, the disposable garment 20 could cause leakage problems when the disposable garment 20 is used alone without an insert pad. Therefore, it is preferable to let the user know the necessity of use of an insert pad when in use of the disposable garment 20. For that purpose, the disposable garment 20 has directions 118 as to the necessity of an insert pad. The directions 118 may be any known means such as a character as shown in FIG. 17 or a graphic. The directions 118 may be printed on a separate sheet which is joined to the disposable garment 20 or be directly printed on the elements constituting the disposable garment 20. The directions 118 may be positioned anywhere on the disposable garment 20 as far as the directions 118 draws the wearer's attention. For example, the directions 118 may be provided on the topsheet such that the user can see the directions when the disposable garment 20 is unfolded or the waist opening is opened for wearing. Alternatively, the directions 118 may be provided on the backsheet such that the user can see the directions when the disposable garment 20 is taken out from the package of the disposable garment 20. In the embodiment shown in FIG. 17, the directions 118 are printed on the topsheet 70 in the center crotch portion (not shown in FIG. 17).

Another embodiment is possible to let the user know the necessity of use of an insert pad when in use of the disposable garment 20 instead of having the directions 118. If the user notices that the absorbent capacity per unit area of the center crotch portion 86 is less than that of the periphery portion 88 (e.g., the disposable garment 20 has no absorbent material or less absorbent material in the center crotch portion), the user would be motivated to use an insert pad. Therefore, the outer cover 40 and the backsheet 66 may comprise a translucent material or a transparent material such that the area of no absorbent material or less amount of absorbent material is noticeable from the outside of the garment 20. The transparent or translucent material may comprise any materials such as a translucent nonwoven or a transparent plastic film. The outer cover 40 and the backsheet 66 being translucent or transparent is also useful to observe the inside condition of the disposable garment without taking the wearer's garment off. It is also useful to combine a disposable garment having an outer cover and/or a backsheet comprising a translucent or transparent material and an inner pad provided with a urine indicator on the garment facing side. It allows the caregiver to observe the timing of the change of the inner pad without taking the wearer's garment and the inner pad off.

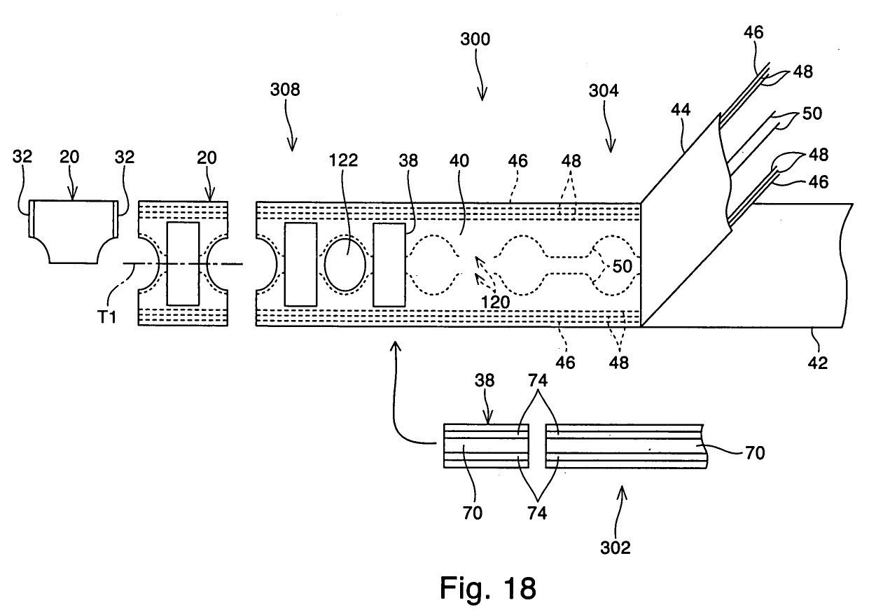

FIG. 18 shows a schematic view to explain a process for forming the disposable garment 20. The process 300 shown in FIG. 18 primarily comprises three sections; an absorbent assembly forming section 302, an outer cover forming section 304 and an assembly section 308.

The absorbent assembly forming section 302 combines elements constituting the absorbent assembly 38 such as the topsheet 70, the backsheet 66 (not shown), the absorbent material 68 (not shown) and the barrier leg cuff 74 including the barrier leg cuff elastic member 76 (i.e., absorbent assembly elastic member 72) (not shown). These elements are joined to each other by any known means such as adhesives. If the absorbent assembly comprises the reinforcement member for stiffness, the reinforcement member may be joined to the absorbent assembly 38 in the absorbent assembly forming section 302. Alternatively, if absorbent material 68 is stiffened by compressing or embossing a portion of or the entirety of the absorbent material 68, the absorbent material 68 may be compressed or embossed before, when or after being joined with other elements such as the topsheet 70 or the backsheet 66. The absorbent assembly forming section 302 cut the continuous assembly into an individual absorbent assembly 38.

The outer cover forming section 304 combines the outer layer 42, the inner layer 44 and the outer cover elastic member comprising the waist end elastic member 46, the waist body elastic member 48 and the leg elastic member 50 (in the embodiment shown in FIG. 18, the number of each elastic member is abbreviated). The waist end elastic member 46 and the waist body elastic member 48 are disposed to form a straight line along the machine direction. The leg elastic member 50 is disposed to form a regular wavy line as shown in FIG. 18. Then the leg elastic member 50 is cut to form the discontinuity 120 at the crotch region 30 of the disposable garment 20 (refer to FIG. 2) to form the continuous outer cover 40.

The assembly section 308 combines the individual absorbent assembly 38 with the continuous outer cover 40. The individual absorbent assembly 38 is turned by 90 degree from the absorbent assembly forming section 302. After the individual absorbent assembly 38 is joined to the continuous outer cover 40, the leg hole 122 is cut from the continuous outer cover 40. Because the stiffened absorbent assembly 38 as described above tends to withstand the contract force of the absorbent assembly elastic member 72, the stiffened absorbent assembly 38 allows to employ an easy process of handling the absorbent assembly 38 when being turned by 90 degree before being joined to the continuous outer cover 40. Further, the stiffened absorbent assembly 38 is kept attached to the outer cover 40 after being joined to the outer cover 40. The continuous outer cover 40 having the absorbent assembly 38 is then cut into the individual disposable garment 20. The individual disposable garment 20 is folded along the transverse centerline T1 and joined at the seam 32 to form a pull-on disposable garment 20.

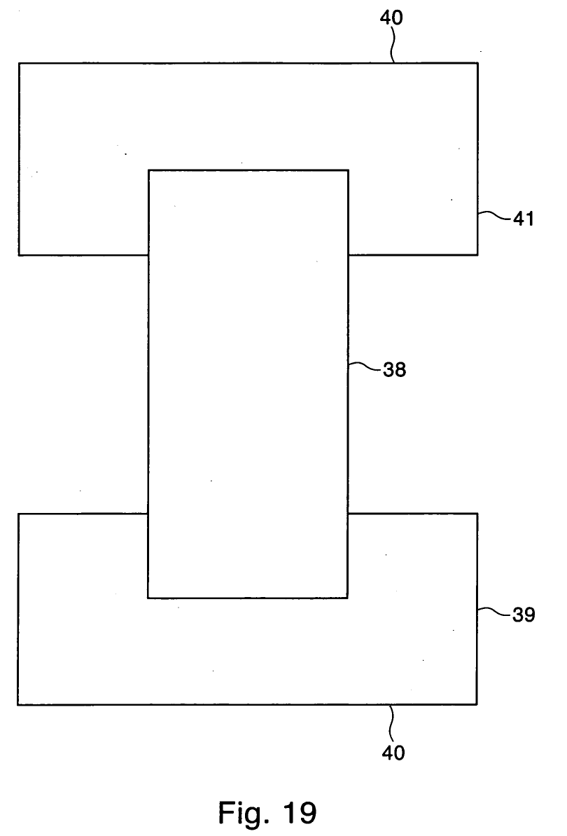

FIG. 19 shows an alternative embodiment of the outer cover. While the outer cover 40 in the embodiment shown in FIGS. 1 and 2 covers the front and back waist region and the crotch region of the wearer, the outer cover 40 in the embodiment shown in FIG. 19 only covers the front and back waist region of the wearer. The outer cover 40 shown in FIG. 19 comprises a front outer cover 39 and a back outer cover 41 and does not extend into the crotch region 30 (refer to FIG. 2). The outer cover 40 may comprise an outer layer, an inner layer and outer cover elastic members disposed therebetween. The absorbent assembly 38 may be placed on and joined inside the outer cover 40 at the portion where the absorbent assembly 38 and the outer cover 40 overlaps. Thus, the absorbent assembly 38 is only partly covered by the outer cover 40.

### Method to Measure Absorbent Capacity Per Unit Area

1. Determination of Center Crotch Portion and Periphery Portion

(1) Separate an absorbent assembly from an outer cover
(2) Stretch an absorbent assembly in its flat uncontracted condition
(3) Determine the longitudinal centerline L2 and the transverse centerline T2 of the absorbent assembly
(4) Measure the total area (TA) of the absorbent assembly
(5) Determine the center crotch portion to have an oval shape with the conditions as follows.
   (a) The oval shape has the length A of the longitudinal axis on the longitudinal centerline L2, the width B of the transverse axis on the transverse centerline T2 and the area X.
   (b) The ratio of A: B is 1.6:1 and the area X is 35% of the total area (TA) of the absorbent assembly.
(6) Measure the area (CA) of the center crotch portion
(7) Measure the area (PA) of the periphery portion
   2. Measurement
(1) Cut the absorbent assembly to separate the center crotch portion from the periphery portion
(2) Seal the cut edge of the center crotch portion and the cut edge of the periphery portion with a tape having no absorbency to prevent the absorbent material from scattering
(3) Put the cut-off center crotch portion into a mesh bag having the mesh size being able to prevent absorbent material from passing through the mesh
(4) Measure the dry weight (CDW) of the bag containing the cut-off center crotch portion
(5) Soak the bag containing the cut-off center portion into a pan filled with a sufficient amount of 0.9% NaCl solution
(6) Wait for ten (10) minutes and pull up the bag from the pan
(7) Suspend the bag for ten (10) minutes to drain excess saline
(8) Measure the wet weight (CWW) of the bag containing the cut-off center crotch portion
(9) Continue the same processes through (3) to (8) for the cut-off periphery portion (the dry weight and wet weight being PDW and PWW, respectively)
   3. Calculation
(1) Calculate the absorbent capacity per unit area for the center crotch portion (CAC) and the absorbent capacity per unit area for the periphery portion (PAC) as follows.
   CAC=(CWW−CDW)/CA
   PAC=(PWW−PDW)/PA

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

What is claimed is:

1. A disposable garment having a longitudinal centerline, a transverse centerline, a crotch region, a front region and a back region, the disposable garment comprising
an outer cover and an absorbent assembly joined inside the outer cover,
the absorbent assembly having a longitudinal centerline, a transverse centerline, longitudinal sides and transverse ends and comprising a backsheet, an absorbent material provided inside the backsheet and an elastic member extending in the longitudinal direction of the absorbent assembly to longitudinally elasticize a portion of the absorbent assembly,
the absorbent assembly having a center crotch portion and a periphery portion, each portion having an absorbent capacity per unit area, the absorbent capacity per unit area of the center crotch portion being less than that of the periphery portion, wherein the absorbent assembly is treated to increase stiffness.

2. The disposable garment of claim 1 wherein the absorbent capacity per unit area of the center crotch portion is not more than 40% of the absorbent capacity per unit area of the periphery portion.

3. The disposable garment of claim 2 wherein the center crotch portion has no absorbent material to have no absorbent capacity.

4. The disposable garment of claim 1 wherein the absorbent assembly has a longitudinally extending center portion and longitudinally extending opposite side portions, the elastic member is positioned in the area of the longitudinally extending side portion when the absorbent assembly is stretched in its flat uncontracted condition, and the longitudinally extending side portion is treated to increase stiffness thereof.

5. The disposable garment of claim 4 wherein the transverse width of the longitudinally extending center portion is not more than 40% of the transverse width between the positions of the elastic members when the absorbent assembly is stretched in its flat uncontracted condition.

6. The disposable garment of claim 5 wherein the absorbent garment comprises a barrier leg cuff disposed adjacent the longitudinal sides of the absorbent assembly, the barrier leg cuff has a proximal edge and a distal edge, and the elastic member to longitudinally elasticize a portion of the absorbent assembly is an elastic member disposed adjacent the distal edge of the barrier leg cuff.

7. The disposable garment of claim 1 wherein the absorbent assembly is incorporated with a reinforcement member to increase stiffness.

8. The disposable garment of claim 1 wherein the absorbent material is treated to increase stiffness.

9. The disposable garment of claim 1 wherein both of the outer cover and the backsheet comprise a translucent material or a transparent material.

10. The disposable garment of claim 1 wherein the absorbent assembly comprises a topsheet to cover at least the absorbent material.
